(19) United States
(12) Patent Application Publication
(10) Pub. No.: US 2026/0115433 A1
(43) Pub. Date: Apr. 30, 2026

(54) BALLOON FOR BALLOON CATHETER, BALLOON CATHETER INCLUDING SAME, AND METHOD FOR MANUFACTURING BALLOON CATHETER

(71) Applicant: KANEKA CORPORATION, Osaka (JP)

(72) Inventors: Masahiro Kojima, Osaka (JP); Takahisa Hamabuchi, Osaka (JP); Masato Tsueda, Osaka (JP)

(73) Assignee: KANEKA CORPORATION, Osaka (JP)

(21) Appl. No.: 19/398,521

(22) Filed: Nov. 24, 2025

Related U.S. Application Data
(63) Continuation of application No. PCT/JP2024/018120, filed on May 16, 2024.

(30) Foreign Application Priority Data
May 30, 2023 (JP) 2023-089133

(51) Int. Cl. A61M 25/10

(52) U.S. Cl. CPC A61M 25/1029; A61M 2025/1086; A61M 2207/00

(57) ABSTRACT

A balloon for a balloon catheter, the balloon having a protruding portion with a desired shape obtained by cutting a predetermined portion of the protruding portion efficiently while preventing damage to other portions, a balloon catheter including the balloon, and a method for manufacturing the balloon catheter are provided.

A balloon for a balloon catheter includes a balloon main body having an outer surface and an inner surface, and a protruding portion protruding outward in a radial direction from the outer surface of the balloon main body and extending in a longitudinal axis direction of the balloon main body, and has a protruding region including the protruding portion in a circumferential direction of the balloon main body and a non-protruding region not including the protruding portion in the circumferential direction. At least a part of the protruding region is colored with a pigment or a dye.

### TECHNICAL FIELD

One or more embodiments of the present invention relate to a balloon for a balloon catheter, a balloon catheter including the same, and a method for manufacturing a balloon catheter.

### BACKGROUND

Angioplasty, in which a balloon catheter is inserted into a stenosed site of a blood vessel and the balloon is inflated to expand the blood vessel and secure blood flow, is widely performed as a minimally invasive therapy.

Angioplasty is used, for example, for the treatment of diseases such as myocardial infarction caused by stenosis occurring in the coronary artery of the heart, the treatment of stenosis occurring in a shunt portion for dialysis, and the like.

In angioplasty, it is sometimes difficult to expand a stenosed site hardened by calcification or the like with a general balloon catheter. In addition, a method of expanding a stenosed site by placing an indwelling expansion instrument called a stent in the stenosed site is also used. However, for example, a lesion such as an in-stent restenosis (ISR) lesion in which neointima of a blood vessel excessively proliferates after the treatment and stenosis of the blood vessel occurs again may occur. In the ISR lesion, since the neointima is soft and the surface thereof is slippery, in a general balloon catheter, the position of the balloon may be shifted from the lesion portion at the time of expanding the balloon, and the blood vessel may be damaged.

As a balloon catheter capable of expanding a stenosed site even in a calcified lesion or an ISR lesion, a balloon catheter in which a protruding portion such as a blade or a scoring element for biting into the stenosed site is provided on a balloon has been developed (for example, PTLs 1 to 3). A balloon provided with such a protruding portion tends to have high rigidity at a portion where the protruding portion is provided, and the bendability in the longitudinal axis direction is likely to decrease. In contrast, a balloon catheter in which a notch is formed in a protruding portion is known (for example, PTLs 4 and 5). When such a balloon catheter is used, it is possible to secure bendability in the longitudinal axis direction even in the case of a balloon provided with a protruding portion.

### Patent Literature

PTL 1: International Publication No. 2020/250611

PTL 2: Japanese Unexamined Patent Application

### Publication No. 2009-112361

PTL 3: Japanese Unexamined Patent Application

### Publication No. 2013-176507

PTL 4: International Publication No. 2012/099950

PTL 5: International Publication No. 2020/255923

A recess such as a notch of a protruding portion provided on the surface of a balloon can be provided, for example, by cutting a part of the protruding portion during or after the manufacture of the balloon. A laser is suitably used for the cutting, but when a high-output laser is used in order to increase the cutting efficiency, a large amount of electric power is required for the balloon manufacture, which decreases manufacture efficiency. In addition, portions other than a portion desired to be cut are easily damaged by the laser, thereby reducing the strength and pressure resistance of the balloon.

### SUMMARY

In view of the above-described circumstances, a balloon for a balloon catheter that is a balloon provided with a protruding portion on a surface thereof and that is capable of being provided with a protruding portion having a desired shape by efficiently cutting a predetermined portion of the protruding portion while preventing damage to other portions, a balloon catheter including the balloon, and a method for manufacturing the balloon catheter are provided.

A balloon for a balloon catheter according to one or more embodiments of the present invention that can address the above is as follows.
[1] A balloon for a balloon catheter, comprising: a balloon main body portion having an outer surface and an inner surface; and a protruding portion protruding outward in a radial direction on the outer surface of the balloon main body portion and extending in a longitudinal axis direction of the balloon main body portion, wherein a protruding region including the protruding portion and a non-protruding region not including the protruding portion are provided in a circumferential direction of the balloon main body portion, and at least a part of the protruding region is colored with a pigment or a dye.

By partially cutting the protruding portion provided in the balloon to provide a notch or a recess, it is possible to improve the function of expanding a stenosed site and the flexibility of the balloon. In this case, a laser can be used for cutting the protruding portion. However, while the balloon for a balloon catheter is typically made of a synthetic resin and thus is colorless, at least a part of the protruding region of the balloon is colored with the pigment or the dye, and thus, the colored portion readily absorbs a laser beam compared with a colorless resin.

Therefore, by appropriately selecting a portion to be colored, a notch or a recess can be provided in a predetermined portion. In addition, since the colored portion readily absorbs even a low-output laser beam compared with a colorless resin, it is possible to efficiently mold the protruding portion in the protruding region into a desired shape. As a result, it is possible to form the protruding portion of the balloon into a shape corresponding to a treatment target, and it is possible to impart an intended function of expanding a stenosed site and flexibility to the balloon.

A balloon for a balloon catheter according to one or more embodiments of the present invention may be any one of the following [2] to [6].
[2] The balloon for a balloon catheter according to [1], wherein the non-protruding region is not colored.
[3] The balloon for a balloon catheter according to [1] or [2], wherein in the cross section perpendicular to the longitudinal axis direction, the balloon main body portion in the protruding region is not colored.
[4] The balloon for a balloon catheter according to any one of [1] to [3], wherein the protruding portion has at least one depressed portion, and the protruding region is continuously colored in the longitudinal axis direction.
[5] The balloon for a balloon catheter according to any one of [1] to [3], wherein the protruding portion has at least one depressed portion, and at least a part of the depressed portion is not colored.
[6] The balloon for a balloon catheter according to any one of [1] to [3] and [5], wherein the protruding portion has at least one depressed portion, and at least a part of the protruding region other than the depressed portion is not colored.

One or more embodiments of the present invention also provide the following.
[7] A balloon catheter comprising the balloon for a balloon catheter according to any one of the above [1] to [6].

One or more embodiments of the present invention also provide the following.
[8] A tubular member which has a lumen extending in a longitudinal axis direction and is used for manufacturing a balloon for a balloon catheter by blow molding, the tubular member comprising: a protruding region which protrudes outward in a radial direction and extends in the longitudinal axis direction; and a non-protruding region other than the protruding region, wherein at least a part of the protruding region is colored with a pigment or a dye.

A tubular member according to one or more embodiments of the present invention may be the following [9] or [10].
[9] The tubular member according to [8], in which the protruding region is continuously colored in the longitudinal axis direction.
[10] The tubular member according to [8], in which the protruding region is intermittently colored in the longitudinal axis direction.

One or more embodiments of the present invention also provide the following.
[11] A method for manufacturing a balloon catheter, the method including: preparing a tubular member having a protruding region protruding outward in a radial direction and extending in a longitudinal axis direction and a non-protruding region other than the protruding region, the protruding region having a colored portion colored with a pigment or a dye in at least a part thereof; cutting at least a part of the colored portion with a laser; and manufacturing a balloon for a balloon catheter by blow molding the tubular member, the balloon having a protruding portion protruding outward in a radial direction and extending in a longitudinal axis direction, the protruding portion having at least one depressed portion.

A method for manufacturing a balloon catheter according to one or more embodiments of the present invention may be the following [12] or [13].
[12] The manufacturing method according to [11], in which the colored portion is continuously disposed in the longitudinal axis direction in the protrusion region.
[13] The manufacturing method according to [11], in which the colored portion is disposed intermittently in the longitudinal axis direction in the protruding region.

### Advantageous Effects of Invention

According to the above-described balloon for a balloon catheter, the balloon catheter including the same, and the method for manufacturing a balloon catheter, since at least a part of the protruding region is colored with a pigment or a dye, the colored portion can efficiently absorb a laser beam. This makes it possible to efficiently cut the colored portion provided at a predetermined position on the protruding portion while preventing damage to other portions, and thus, it is possible to easily provide a balloon for a balloon catheter including a protruding portion having a desired shape.

### BRIEF DESCRIPTION OF DRAWINGS

FIG. 1 is a side view of a balloon catheter according to one or more embodiments of the present invention.

FIG. 2 is a cross-sectional view of the balloon catheter taken along line II-II of FIG. 1.

FIG. 3 is a longitudinal sectional view of a balloon of the balloon catheter illustrated in FIG. 1.

FIG. 4 is a sectional view illustrating a modification of FIG. 3.

FIG. 5 is a cross-sectional view perpendicular to the longitudinal axis direction of the balloon illustrated in FIG. 3.

FIG. 6 is a cross-sectional view perpendicular to the longitudinal axis direction of the balloon illustrated in FIG. 4.

FIG. 7 is a cross-sectional view illustrating a modification of FIG. 6.

FIG. 8 is a perspective view of a balloon according to one or more embodiments of the present invention.

FIG. 9 is a longitudinal sectional view of the balloon illustrated in FIG. 8.

FIG. 10 is a sectional view illustrating a modification of FIG. 9.

FIG. 11 is a plan view of the balloon illustrated in FIG. 9 as viewed from a protruding portion side.

FIG. 12 is a sectional view illustrating a further modification of FIG. 9.

FIG. 13 is a plan view of the balloon illustrated in FIG. 12 as viewed from the protruding portion side.

FIG. 14 is a sectional view illustrating a further modification of FIG. 9.

FIG. 15 is a sectional view illustrating a further modification of FIG. 9.

FIG. 16 is a perspective view of a tubular member according to one or more embodiments of the present invention.

FIG. 17 is a cross-sectional view of the tubular member taken along line XVII-XVII of FIG. 16.

FIG. 18 is a plan view of the tubular member illustrated in FIG. 17 as viewed from a protruding region side.

FIG. 19 is a plan view illustrating a modification of FIG. 18.

### DETAILED DESCRIPTION

Hereinafter, one or more embodiments of the present invention will be described on the basis of embodiments, but the present invention is not limited to the following embodiments, and it is of course possible to carry out the present invention by adding appropriate modifications within a range that can be adapted to the gist described above and below, and all of them are included in the technical scope of the present invention. In each drawing, hatching, the reference signs of components, and the like may sometimes be omitted for convenience. In such cases, reference may be made to the specification or the other drawings. Additionally, the dimensions of various components in the drawings may differ from the actual dimensions as priority is given to facilitating understanding of the features of one or more embodiments of the present invention.

### 1. Balloon for Balloon Catheter

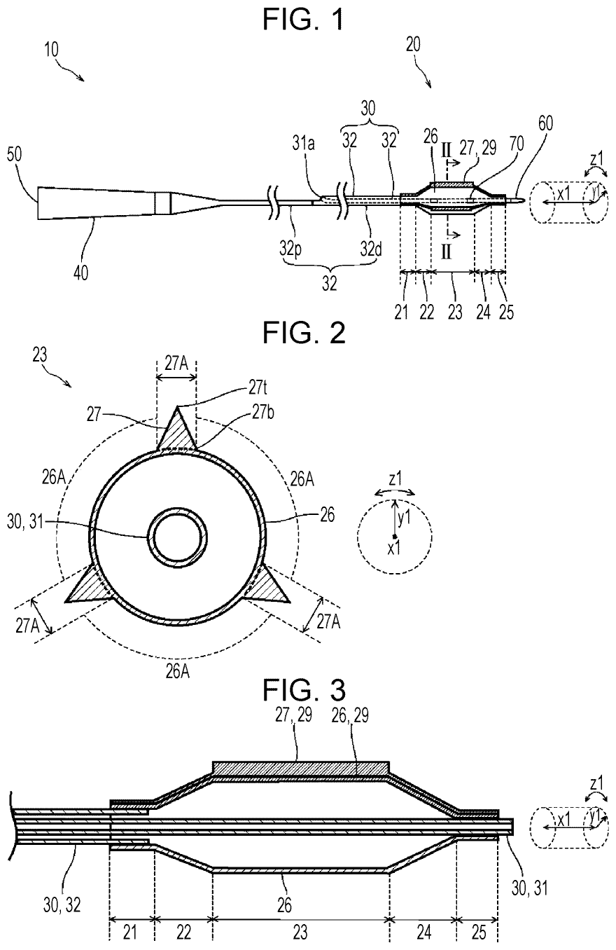

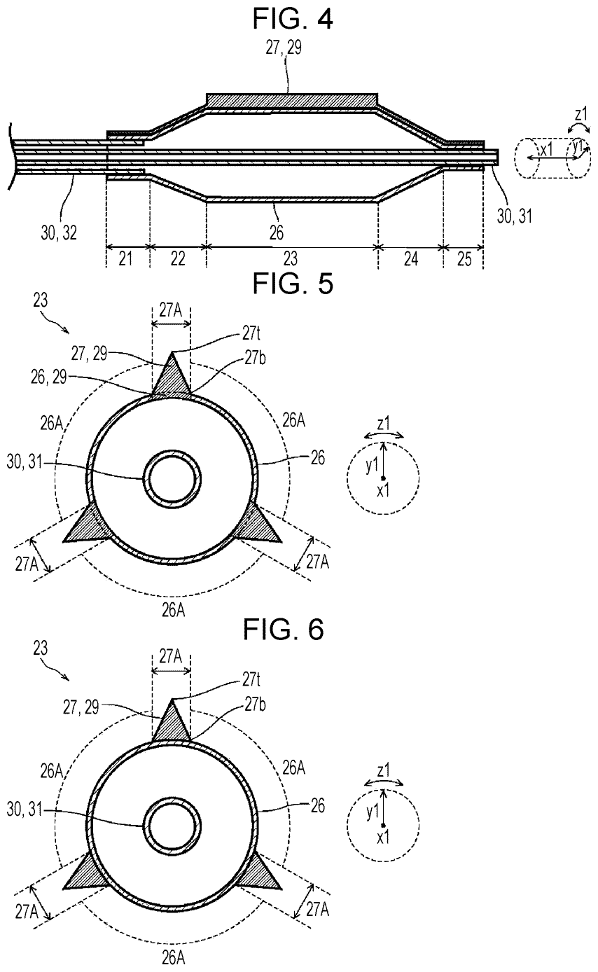

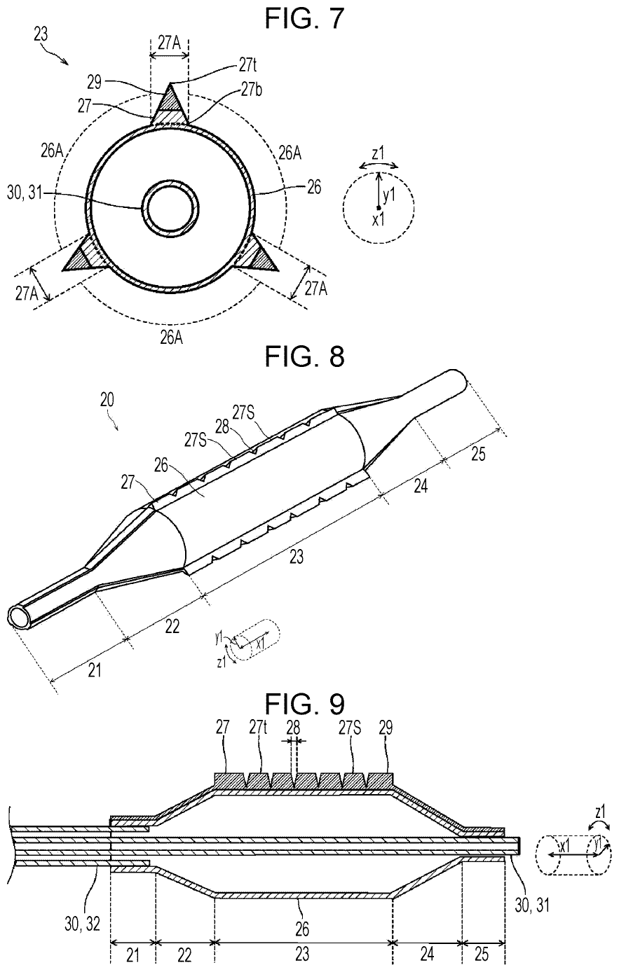

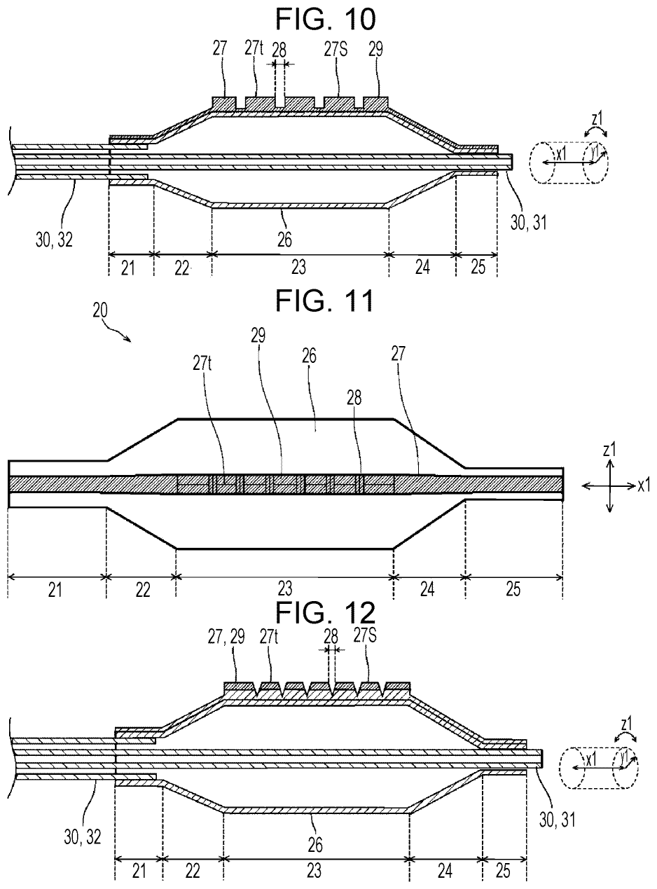

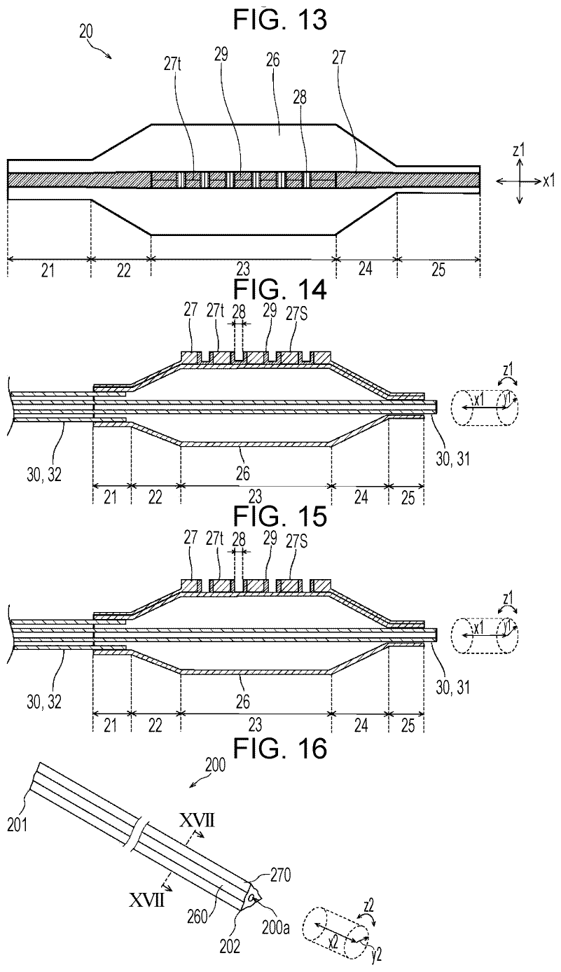

A balloon for a balloon catheter according to one or more embodiments of the present invention will be described with reference to FIGS. 1 to 15. FIG. 1 is a side view of a balloon catheter according to one or more embodiments of the present invention. Hatching in FIG. 1 represents a colored portion. FIG. 2 is a cross-sectional view of the balloon catheter taken along line II-II of FIG. 1. The colored portions are not distinguished in FIG. 2. FIG. 3 is a longitudinal sectional view of a balloon of the balloon catheter illustrated in FIG. 1 and illustrates a cross section passing through an apex of a protruding portion and the central axis of the balloon. FIG. 4 is a sectional view illustrating a modification of FIG. 3. FIG. 5 is a cross-sectional view perpendicular to the longitudinal axis direction of the balloon illustrated in FIG. 3, and FIG. 6 is a cross-sectional view perpendicular to the longitudinal axis direction of the balloon illustrated in FIG. 4. FIG. 7 is a cross-sectional view illustrating a modification of FIG. 6. FIG. 8 is a perspective view of a balloon according to one or more embodiments of the present invention. FIG. 9 is a longitudinal sectional view of the balloon illustrated in FIG. 8 and illustrates a cross section passing through an apex of a protruding portion and the central axis of the balloon. FIG. 10 is a sectional view illustrating a modification of FIG. 9. FIG. 11 is a plan view of the balloon illustrated in FIG. 9 as viewed from the protruding portion side. Hatching in FIG. 11 does not represent a cross section, but represents a colored portion. FIG. 12 is a sectional view illustrating a further modification of FIG. 9. FIG. 13 is a plan view of the balloon illustrated in FIG. 12 as viewed from the protruding portion side. Hatching in FIG. 13 does not represent a cross section, but represents a colored portion. FIGS. 14 and 15 are sectional views illustrating different modifications of FIG. 9. In the present specification, a balloon for a balloon catheter may be simply referred to as a “balloon”.

As illustrated in FIG. 1, a balloon 20 is provided at a distal portion of a balloon catheter 10. The balloon 20 is connected to a distal end portion of a shaft 30, and the balloon 20 can be inflated by introducing a fluid therein through a lumen of the shaft 30, and the balloon 20 can be deflated by discharging the fluid. In order to control the inflation and deflation of the balloon 20, a fluid can be introduced into and discharged from the balloon 20 by using an indeflator (balloon pressurizer). The fluid may be a pressurized fluid that is pressurized by a pump or the like. The balloon catheter 10 will be described in detail in the section “2. Balloon Catheter”.

As illustrated in FIGS. 1 to 12, the balloon 20 has a longitudinal axis direction x1, a radial direction y1 that is a direction connecting a centroid of a circumscribed circle of the balloon 20 and a point on the circumscribed circle in a cross section perpendicular to the longitudinal axis direction x1, and a circumferential direction z1 which is a direction along the circumscribed circle. In the present specification, a direction toward a user's hand in the longitudinal axis direction x1 is referred to as a proximal side, and a side opposite to the proximal side, that is, a direction toward a treatment target person is referred to as a distal side.

Members and portions of the balloon catheter 10 other than the balloon 20 each have a longitudinal axis direction, a radial direction, and a circumferential direction, which may be the same as or different from the longitudinal axis direction x1, the radial direction y1, and the circumferential direction z1 of the balloon 20, respectively. However, in the present specification, for ease of understanding, each member and portion is described as having the same longitudinal axis direction, radial direction, and circumferential direction as the longitudinal axis direction x1, the radial direction y1, and the circumferential direction z1 of the balloon 20.

As illustrated in FIGS. 1 and 2, the balloon 20 according to one or more embodiments of the present invention has a balloon main body 26 having an outer surface and an inner surface, and at least one protruding portion 27 protruding outward in the radial direction y1 from the outer surface of the balloon main body 26 and extending in the longitudinal axis direction x1 of the balloon main body 26. The balloon 20 has at least one protruding region 27A and a non-protruding region 26A, the protruding region 27A including the protruding portion 27 and the non-protruding region 26A not including the protruding portion 27 in the circumferential direction z1 of the balloon main body 26.

The protruding region 27A may have a length of the maximum width of the protruding portion 27 in the circumferential direction z1 of the balloon main body 26. The balloon main body 26 is a portion that defines the basic shape of the balloon 20. The protruding portion 27 can be rephrased as a portion formed thicker than the non-protruding region 26A, that is, a portion where the balloon main body 26 is exposed.

The thickness of the protruding region 27A of the balloon 20 may be 1.2 times or more, 1.5 times or more, 1.8 times or more, 2.0 times or more, or 2.5 times or more the thickness of the non-protruding region 26A of the balloon 20. The upper limit of the thickness of the protruding region 27A of the balloon 20 is not particularly limited, and may be, for example, 30 times or less, 20 times or less, or 10 times or less the thickness of the non-protruding region 26A of the balloon 20.

In the circumferential direction z1, a portion of the entire circumference of the balloon 20 other than the protruding region 27A is the non-protruding region 26A, and it is preferable that irregularities not be formed on the non-protruding region 26A. The thickness of the balloon main body 26 in the non-protruding region 26A may be uniform in the longitudinal axis direction x1 and the circumferential direction z1. Here, the term “irregularities” does not include surface roughness which is inevitably formed in manufacturing. Thus, the balloon 20 can be easily inflated uniformly, and the scoring function of the protruding portion 27 can be easily exerted as desired.

Since the balloon 20 has the protruding portion 27, a scoring function can be imparted to the balloon 20, and by bringing the protruding portion 27 into contact with a lumen wall of a stenosed site and making the protruding portion 27 bite into the lumen wall, even a calcified lumen wall of the stenosed site can be cracked. Therefore, it is possible to expand a stenosed site while suppressing the dissection of a vascular intima. In addition, since the protruding portion 27 can improve the rigidity of the balloon 20 due to the thickness and the rigidity thereof, it is also possible to increase the pressure resistance of the balloon 20 and to suppress excessive inflation of the balloon 20 during pressurization.

As illustrated in FIG. 2, the protruding portion 27 may have a top portion 27t and a base portion 27b. The top portion 27t is a portion including an outer end of the protruding portion 27 in the radial direction y1, and the base portion 27b is a portion including a boundary with the balloon main body 26, that is, an inner end of the protruding portion 27 in the radial direction y1. By making the top portion 27t bite into a lumen wall of a stenosed site, a crack can be easily formed.

As illustrated in FIGS. 3 and 4, the protruding portion 27 may be provided on the outer surface of the balloon main body 26 so as to extend in a ridge-like manner in the longitudinal axis direction x1. As illustrated in FIG. 2, a plurality of protruding portions 27 may be provided in the circumferential direction z1, or, although not illustrated, only one protruding portion 27 may be provided in the circumferential direction z1. The number of the protruding portions 27 in the circumferential direction z1 may be 1 or more, 2 or more, 3 or more, 4 or more, or 6 or more, and may be 20 or less, 15 or less, or 10 or less. In the case where a plurality of protruding portions 27 are provided in the circumferential direction z1, the plurality of protruding portions 27 may be separated from each other in the circumferential direction z1, and may be arranged at substantially equal intervals in the circumferential direction z1. The distance between the protruding portions 27 separated from each other may be longer than the maximum length of the protruding regions 27A in the circumferential direction z1.

The cross-sectional shape of the protruding portion 27 in a cross section perpendicular to the longitudinal axis direction x1 may be any shape, and may be, for example, a triangle, a quadrangle, a polygon, a semicircle, a part of a circle, a substantially circular shape, a fan shape, a wedge shape, a convex-character shape, a spindle shape, or a combination thereof. Note that the terms “triangle”, “quadrangle”, and “polygon” include not only those with clearly defined vertices and straight sides, but also so-called rounded-corner polygons with rounded corners, and shapes in which at least a part of side portions is curved. Alternatively, the cross-sectional shape of the protruding portion 27 may be an irregular shape having irregularities, chips, or the like. The cross-sectional shape of the protruding portion 27 may vary depending on the position in the longitudinal axis direction x1.

As illustrated in FIG. 1, the balloon 20 for a balloon catheter according to one or more embodiments of the present invention may have the straight tube portion 23, a proximal-side tapered portion 22 located on the proximal side of the straight tube portion 23, a proximal-side sleeve portion 21 located on the proximal side of the proximal-side tapered portion 22, a distal-side tapered portion 24 located on the distal side of the straight tube portion 23, and a distal-side sleeve portion 25 located on the distal side of the distal-side tapered portion 24.

The proximal-side tapered portion 22 and the distal-side tapered portion 24 may be formed such that the diameter of a balloon main body 26 decreases as the distance from the straight tube portion 23 increases. With such a configuration, the straight tube portion 23 of the balloon 20 can have the maximum diameter when the balloon 20 is in an inflated state, and by inflating the balloon 20 at a lesion portion, the straight tube portion 23 can be brought into sufficient contact with the lesion portion to facilitate expansion and cutting of a stenosed site. In addition, since the balloon 20 includes the proximal-side tapered portion 22 and the distal-side tapered portion 24, when the balloon 20 is deflated, the outer diameter of a proximal end portion of the balloon 20 and the outer diameter of a distal end portion of the balloon 20 can be reduced so as to reduce the difference in diameter between the shaft 30 and the balloon 20, so that it becomes easier to insert the balloon 20 into a body cavity, a forceps channel of an endoscope, or a delivery catheter such as a guiding catheter.

The proximal-side sleeve portion 21 and the distal-side sleeve portion 25 may be portions that do not inflate even when the balloon 20 is in the inflated state. Accordingly, at least a part of the proximal-side sleeve portion 21 and at least a part of the distal-side sleeve portion 25 can be configured to be stably fixed to the shaft 30. As will be described later, in the case of a configuration in which the shaft 30 includes an inner shaft 31 and an outer shaft 32, at least a part of the proximal-side sleeve portion 21 can be fixed to the outer shaft 32, and at least a part of the distal-side sleeve portion 25 can be fixed to the inner shaft 31.

The protruding portion 27 may be provided in the straight tube portion 23. A range in which the protruding portion 27 is disposed in the longitudinal axis direction x1 may be the entire section of the straight tube portion 23. Alternatively, the range in which the protruding portion 27 is disposed in the longitudinal axis direction x1 may be a partial section of the straight tube portion 23. By providing the protruding portion 27 in the straight tube portion 23 that has the maximum diameter when the balloon 20 is inflated, the function of expanding a stenosed site can be improved.

The protruding portion 27 may be provided in a tapered portion or a sleeve portion. When the protruding portion 27 is provided in the tapered portion or the sleeve portion, the height of the protruding portion 27 in the tapered portion or the sleeve portion may be lower than the height of the protruding portion 27 in the straight tube portion 23. Thus, the maneuverability of the balloon 20 can be improved.

As illustrated in FIGS. 3 to 7, at least a part of the protruding region 27A may be colored with a pigment or a dye. A known pigment or dye can be used. For example, the pigment can be selected from pigments classified as pigments in the Color Index (published by The Society of Dyers and Colourists), and examples thereof include yellow pigments, orange pigments, red pigments, blue pigments, purple pigments, and green pigments. Specific examples of the pigment include amorphous carbon such as carbon black; metal compounds such as titanium oxide, iron oxide (black iron oxide, yellow iron oxide, red iron oxide), chromium oxide, ultramarine (ultramarine blue, ultramarine violet), nickel titanium yellow, Prussian blue, Milori blue, cobalt blue, viridian, molybdenum red, lead, barium, barium sulfate, tungsten, stainless steel, titanium, cobalt-chromium alloy, palladium, bismuth, and tantalum; and inorganic pigments such as iodine. Further examples of the pigment include organic pigment components such as quinacridone pigments (e.g., quinacridone red), perylene pigments (e.g., perylene red), anthraquinone pigments (e.g., anthraquinone yellow), azo pigments (e.g., condensed azo yellow pigments), phthalocyanine pigments (e.g., halogenated phthalocyanines such as copper phthalocyanine and copper phthalocyanine chloride), as well as pigment violet and pigment yellow. In particular, amorphous carbon such as dark-colored carbon black, ultramarine (ultramarine blue and ultramarine violet), Prussian blue, Milori blue, cobalt blue, and viridian, as well as high-density iron oxides (black iron oxide), chromium oxide, molybdenum red, lead, tungsten, stainless steel, titanium, cobalt-chromium alloy, palladium, bismuth, and tantalum are more preferable. The dye can be selected from known dyes described in the Color Index (published by The Society of Dyers and Colourists) or Some-iro Note (published by Shikisensha). One of these may be used, or a combination of two or more of these may be used. The pigment and the dye may be appropriately selected so as to exhibit a color that readily absorbs the wavelength of the laser beam used for cutting the protruding portion 27.

A region of the protruding portion 27 where a colored portion 29 is provided may be the entirety of the protruding portion 27 as illustrated in FIG. 6 or may be a part of the protruding portion 27 as illustrated in FIG. 7. Specifically, an example of such a configuration is one in which a portion including the top portion 27t of the protruding portion 27 is colored, in which a portion including the base portion 27b is not colored, and in which a part of the protruding region 27A in the circumferential direction z1 is colored. Further, although not particularly illustrated, the colored portion 29 may be provided only on the surface side of the protruding portion 27, and the inside of the protruding portion 27 may not be colored.

As will be described later, by partially cutting the protruding portion 27 to provide at least one recess 28, it is possible to improve the function of expanding a stenosed site by the balloon 20 and the flexibility of the balloon 20. In this case, a laser can be used for cutting the protruding portion 27. Since the balloon 20 is typically manufactured from a synthetic resin and thus is colorless, the colored portion 29 colored with the pigment or the dye readily absorbs a laser beam compared with a colorless resin. Therefore, by appropriately selecting the position at which the colored portion 29 is disposed, it is possible to provide the recess 28 at a predetermined portion by irradiating the colored portion 29 with the laser beam to cut the colored portion 29. In addition, the colored portion 29 readily absorbs even a low-output laser beam compared with a colorless resin, and thus, the protruding portion 27 in the protruding region 27A can be efficiently formed into a desired shape. As a result, it is possible to provide the balloon 20 with a predetermined function of expanding a stenosed site and flexibility depending on a treatment target.

As illustrated in FIGS. 3 and 5, in the cross section perpendicular to the longitudinal axis direction x1, the entire portion of the protruding region 27A in the radial direction y1, that is, both the balloon main body 26 and the protruding portion 27 in the protruding region 27A may be colored. This makes it easy to manufacture the balloon 20 from a tubular member made of a resin.

Alternatively, as illustrated in FIGS. 4 and 6, in the cross section perpendicular to the longitudinal axis direction x1, it is preferable that the balloon main body 26 in the protruding region 27A not be colored. In this case, while the protruding portion 27 in the protruding region 27A is colored and easily cut by a laser beam, the balloon main body 26 is less likely to be damaged by the laser beam. Therefore, even when the protruding portion 27 in the protruding region 27A is cut, it is possible to prevent the film thickness at that portion from being reduced, thereby securing the strength and pressure resistance of the balloon 20.

In this case, as illustrated in FIG. 6, the entire protruding portion 27 may be the colored portion 29, or, as illustrated in FIG. 7, a portion including the base portion 27b of the protruding portion 27 may not be colored, and only a distal end portion of the protruding portion 27 including the top portion 27t may be colored. The depth to which the protruding portion 27 is cut can be adjusted by controlling how deep the colored portion 29 is provided, when viewed from the top portion 27t of the protruding portion 27.

As illustrated in FIGS. 5 to 7, the non-protruding region 26A may not be colored. In this case, the non-protruding region 26A is less likely to be damaged by a laser beam, and it becomes easy to obtain the balloon 20 with a uniform film thickness and without defects.

As illustrated in FIGS. 8 to 10, the protruding portion 27 may have the at least one recess 28. The recess 28 can be formed by cutting the top portion 27t of the protruding portion 27 extending in the longitudinal axis direction x1.

Since the protruding portion 27 has the recess 28, the flexibility of the balloon 20 can be enhanced. Thus, the balloon 20 can be easily inserted even into a bent body cavity. In addition, the protruding portion 27 can be divided into protruding portion segments 27S by the recess 28, and the protruding portion segments 27S act on a lumen wall of a stenosed site, so that the function of expanding the stenosed site can be improved.

The depth of the recess 28 may be 50 μm or more, 70 μm or more, or 100 μm or more. When the lower limit of the depth of the recess 28 is within the above range, it becomes easy to improve the flexibility of the balloon 20 and the function of expanding a stenosed site. The upper limit of the depth of the recess 28 is the height of a portion of the protruding portion 27, the portion being located at the position where the recess 28 is provided, and the depth of the recess 28 may be, for example, 500 μm or less, 400 μm or less, or 300 μm or less. When the upper limit of the depth of the recess 28 is within the above range, it becomes easy to secure the rigidity of the balloon 20.

The shape of the recess 28 is not particularly limited. In a cross section in the longitudinal axis direction x1 passing through the top portion 27t of the protruding portion 27 and the central axis of the balloon main body 26, the shape of the recess 28 may be, for example, a V-shaped notch as illustrated in FIGS. 8 and 9, a shape obtained by removing one side of a rectangle as illustrated in FIG. 10, a U-shape, an irregular shape, or a combination of these shapes.

As illustrated in FIGS. 9 to 11, the protruding portion 27 may have the at least one recess 28, and the protruding region 27A may be colored continuously in the longitudinal axis direction x1. Since the colored portion 29 continuous in the longitudinal axis direction x1 is provided in the protruding region 27A, the recess 28 can be formed at any position.

The recess 28 may be provided only on the top portion 27t side of the protruding portion 27 in the radial direction y1, and the balloon main body 26 may not be exposed through the recess 28. In this case, as illustrated in FIGS. 9 and 10, the bottom of the recess 28 may be colored. Alternatively, as illustrated in FIGS. 12 and 13, at least a part of the recess 28, which is, for example, the bottom portion, may not be colored. In the case illustrated in FIGS. 12 and 13, the colored portion 29 is provided along the longitudinal axis direction x1 except for the bottom of the recess 28.

Alternatively, as illustrated in FIG. 15, the recess 28 may be provided in the entire protruding portion 27 in the radial direction y1, so that the protruding portion 27 may be discontinuously formed along the longitudinal axis direction x1. That is, the protruding portion 27 may have a missing portion extending in the longitudinal axis direction x1, and the balloon main body 26 may be exposed through the missing portion. In this case, the exposed portion of the balloon main body 26 may be colored or may not be colored.

As illustrated in FIGS. 14 and 15, the protruding portion 27 may have the at least one recess 28, and at least a part of the protruding region 27A other than the recess 28 may not be colored. Since a non-colored portion is present in the protruding region 27A, only the colored portion 29 can be cut without cutting the non-colored portion, and the recess 28 can be easily formed at a predetermined position on the protruding portion 27.

The recess 28 may be formed in the protruding portion 27 in the straight tube portion 23. Since the straight tube portion 23 is a long portion of the balloon 20 in the longitudinal axis direction x1, the flexibility of the balloon 20 can be more easily improved by forming the recess 28 in the protruding portion 27 in the straight tube portion 23. In addition, the protruding portion 27 in the straight tube portion 23 is a portion that can most effectively act on a lumen wall of a stenosed site when the balloon 20 is inflated, and thus, it is possible to further improve the function of expanding the stenosed site by forming the recess 28 in the protruding portion 27 in the straight tube portion 23 to form the protruding portion segments 27S.

The number of the recesses 28 formed in the protruding portion 27 in the straight tube portion 23 is not particularly limited, but can be, for example, 1 or more, 3 or more, or 5 or more, and 20 or less, 16 or less, 12 or less, or 8 or less.

The distal-side tapered portion 24 may be provided with the protruding portion 27, and the recess 28 may be formed in the protruding portion 27 in the distal-side tapered portion 24. The protruding portion 27 provided in the distal-side tapered portion 24 can be exposed and can act on a lumen wall even when the balloon 20 is in a deflated state, and thus, a stenosed site can be cut while advancing the balloon 20 in the deflated state (inching the balloon 20 forward) while slippage is prevented by the recess 28.

The proximal-side sleeve portion 21, the proximal-side tapered portion 22, and the distal-side sleeve portion 25 may not be provided with the recess 28. As a result, the protruding portion segments 27S will not become caught by a lumen wall, and thus, the maneuverability of the balloon 20 can be improved. Alternatively, the recess 28 may also be provided in the proximal-side sleeve portion 21, the proximal-side tapered portion 22, and the distal-side sleeve portion 25.

As described above, the recess 28 can be provided at any position on the protruding portion 27 along the longitudinal axis direction x1. However, since the balloon 20 has the colored portion 29, the recess 28 can be easily formed at any position by adjusting the position of the colored portion 29. This makes it easy to configure the balloon 20 to have a predetermined shape.

Portions of the balloon 20 other than the straight tube portion 23, which are, for example, the proximal-side sleeve portion 21, the proximal-side tapered portion 22, the distal-side tapered portion 24, and the distal-side sleeve portion 25, may not be provided with the protruding portion 27, or may be provided with another protruding portion 27 lower than the protruding portion 27 provided in the straight tube portion 23. In this case, the balloon 20 may include, in portions thereof other than the straight tube portion 23, which are, for example, the proximal-side sleeve portion 21, the proximal-side tapered portion 22, the distal-side tapered portion 24, and the distal-side sleeve portion 25, an inner protruding portion protruding inward in the radial direction y1 and extending in the longitudinal axis direction x1 on the inner surface of the balloon main body 26. The inner protruding portion may be disposed at the same position as the protruding portion 27 in the longitudinal axis direction x1 or the circumferential direction z1. It is preferable that the inner protruding portion and the protruding portion 27 be integrally formed, and in this case, a part of the balloon 20 may be formed to have an increased thickness.

The balloon main body 26 may be made of a resin. The resin may be a thermoplastic resin. This makes it easy to manufacture the balloon 20 by molding. Examples of the resin forming the balloon main body 26 include polyolefin-based resins such as polyethylene, polypropylene, and ethylene-propylene copolymers, polyester-based resins such as polyethylene terephthalate and polyester elastomers, polyurethane-based resins such as polyurethane and polyurethane elastomers, polyphenylene sulfide resins, polyamide-based resins such as polyamides and polyamide elastomers, fluororesins, silicone-based resins, and natural rubbers such as latex. Only one of these may be used, or two or more of these may be used in combination. In particular, polyamide-based resins, polyester-based resins, and polyurethane-based resins are suitably used. In particular, it is preferable to use an elastomer resin from the viewpoint of thinning and flexibility of the balloon 20. Examples of a material that is suitable for the balloon 20 among the polyamide-based resins include nylon 12 and nylon 11, and nylon 12 is suitably used from the viewpoint of being relatively easily moldable during blow molding. In addition, from the viewpoint of thinning and flexibility of the balloon 20, polyamide elastomers such as polyether-ester-amide elastomer and polyamide ether elastomer may be used. In particular, polyether-ester-amide elastomer may be used from the viewpoint of high yield strength and favorable dimensional stability of the balloon 20.

The protruding portion 27 can be made of, for example, a resin. When the protruding portion 27 is made of a resin, the protruding portion 27 and the balloon main body 26 may be made of the same resin, and the protruding portion 27 and the balloon main body 26 may be integrally formed. That is, instead of the protruding portion 27 being attached to the outer side surface of the balloon main body 26, it is preferable that a thin portion of the balloon 20 form the balloon main body 26 and that a thick portion of the balloon 20 form the protruding portion 27. The balloon main body 26 may have an inner layer and an outer layer. In this case, the protruding portion 27 may be made of the same resin as that of the outer layer of the balloon main body 26. With such a configuration, the protruding portion 27 is less likely to unintentionally fall off from the balloon main body 26. Alternatively, the protruding portion 27 and the balloon main body 26 may be formed of different resins as long as the resin forming the protruding portion 27 and the resin forming the balloon main body 26 have a certain degree of compatibility. The protruding portion 27 may be attached as a separate member to the outer side surface of the balloon main body 26 by means of welding, adhesive bonding, or the like.

The pigment or the dye may be mixed beforehand in a resin-made tubular member (described later) that is used for forming the balloon 20 or may be applied to at least a part of the protruding region 27A after formation of the balloon 20.

The protruding portion 27 may be made of a metal or a combination of a metal and a resin. The protruding portion 27 may be attached as a separate member to the outer side surface of the balloon main body 26 by means of welding, adhesive bonding, or the like.

### 2. Balloon Catheter

The balloon catheter 10 according to one or more embodiments of the present invention includes the above-described balloon 20 for a balloon catheter. As described in the above section “1. Balloon for Balloon Catheter”, the balloon 20 is connected to the distal end portion of the shaft 30 as illustrated in FIG. 1.

FIG. 1 illustrates the so-called rapid-exchange balloon catheter 10 that includes a guidewire port 31a formed at an intermediate portion of the shaft 30 in a direction from the distal side toward the proximal side, and an inner shaft 31 serving as a guidewire passage extending from the guidewire port 31a to the distal side of the shaft 30.

The shaft 30 may have a fluid flow path and the guidewire passage therein. In order for the shaft 30 to have the fluid flow path and the guidewire passage therein, for example, the shaft 30 may be configured such that it includes the inner shaft 31 and the outer shaft 32 disposed outside the inner shaft 31, the inner shaft 31 functioning as the guidewire passage, and such that a space between the outer shaft 32 and the inner shaft 31 functions as the fluid flow path. In the case of such a configuration, it is preferable that the inner shaft 31 extend to the distal side by penetrating the balloon 20 such that the distal side of the balloon 20 is connected to the inner shaft 31 and such that the proximal side of the balloon 20 is connected to the outer shaft 32.

The balloon catheter 10 may have a distal-side outer shaft 32d and a proximal-side outer shaft 32p. The distal-side outer shaft 32d and the proximal-side outer shaft 32p may be separate members, and a proximal end portion of the distal-side outer shaft 32d may be connected to a distal end portion of the proximal-side outer shaft 32p so as to constitute the outer shaft 32 extending from the balloon 20 to a proximal end portion of the balloon catheter 10. Alternatively, the single outer shaft 32 may extend from the balloon 20 to the proximal end portion of the balloon catheter 10, and the distal-side outer shaft 32d or the proximal-side outer shaft 32p may further be formed of a plurality of tube members.

The shaft 30 may be made of a resin, a metal, or a combination of a resin and a metal. By using a resin as the material forming the shaft 30, it becomes easy to impart flexibility and elasticity to the shaft 30. In addition, by using a metal as the material forming the shaft 30, the deliverability of the balloon catheter 10 can be improved.

Examples of a resin forming the shaft 30 include polyamide-based resins, polyester-based resins, polyurethane-based resins, polyolefin-based resins, fluororesins, vinyl chloride-based resins, silicone-based resins, natural rubbers, and synthetic rubbers. Only one of these may be used, or two or more of these may be used in combination. Examples of a metal forming the shaft 30 include stainless steels such as SUS 304 and SUS 316, platinum, nickel, cobalt, chromium, titanium, tungsten, gold, Ni-Ti alloys, Co-Cr alloys, or combinations thereof.

In the case where the shaft 30 includes the distal-side outer shaft 32d and the proximal-side outer shaft 32p, which are separate members, for example, the distal-side outer shaft 32d can be made of a resin, and the proximal-side outer shaft 32p can be made of a metal. Further, the shaft 30 may have a multilayer structure made of different materials or the same material.

The balloon 20 and the shaft 30 may be joined to each other by, for example, bonding with an adhesive, welding, or crimping with a ring-shaped member attached to a portion where an end portion of the balloon 20 and the shaft 30 overlap each other. In particular, the balloon 20 and the shaft 30 may be joined to each other by welding. By welding the balloon 20 and the shaft 30 to each other, even when the balloon 20 is repeatedly inflated or deflated, the joint between the balloon 20 and the shaft 30 is less likely to be released, and the joint strength can be improved.

A distal end portion of the balloon catheter 10 may be provided with a tip member 60. The tip member 60 may be provided at the distal end portion of the balloon catheter 10 by being connected to a distal end portion of the balloon 20 as a separate member from the inner shaft 31, or a portion of the inner shaft 31 that extends further toward the distal side than the distal end portion of the balloon 20 may function as the tip member 60.

A radiopaque marker 70 may be disposed at a position on the shaft 30 where the balloon 20 is located in the longitudinal axis direction x1, in order to enable confirmation of the position of the balloon 20 under X-ray fluoroscopy. For example, the radiopaque marker 70 can be disposed on the inner shaft 31 disposed inside the balloon 20. The radiopaque marker 70 may be disposed at positions corresponding to both ends of the straight tube portion 23 of the balloon 20, and may alternatively be disposed at a position corresponding to the center of the straight tube portion 23 of the balloon 20.

A hub 40 may be provided on the proximal side of the shaft 30, and the hub 40 may be provided with a fluid injection portion 50 that communicates with the flow path of the fluid to be supplied to the inside of the balloon 20.

The shaft 30 and the hub 40 are joined to each other by, for example, bonding with an adhesive or welding. In particular, the shaft 30 and the hub 40 may be bonded to each other by adhesive bonding. Since the shaft 30 and the hub 40 are bonded to each other, for example, in the case where a material forming the shaft 30 and a material forming the hub 40 are different from each other, such as the case where the shaft 30 is formed of a material having high flexibility while the hub 40 is formed of a material having high rigidity, the bonding strength between the shaft 30 and the hub 40 can be increased, thereby improving the durability of the balloon catheter 10.

Although not illustrated, one or more embodiments of the present invention can also be applied to a so-called over-the-wire balloon catheter having a guidewire passage extending from the distal side to the proximal side of a shaft. In the case of an over-the-wire balloon catheter, it is preferable that an inflation lumen and a guidewire lumen extend to a hub disposed on the proximal side and that proximal-side openings of the respective lumens be provided in the hub having a bifurcated structure.

In the case of a rapid-exchange catheter, it is preferable that the outer wall of the distal-side outer shaft 32d and/or the outer wall of the proximal-side outer shaft 32p be appropriately coated, and it is more preferable that both the distal-side outer shaft 32d and the proximal-side outer shaft 32p be coated. In the case of an over-the-wire catheter, it is preferable that an outer wall of an outer shaft be appropriately coated.

The coating can be a hydrophilic coating or a hydrophobic coating depending on the purpose, and can be applied by immersing the shaft 30 in a hydrophilic coating agent or a hydrophobic coating agent, applying a hydrophilic coating agent or a hydrophobic coating agent to the outer wall of the shaft 30, or covering the outer wall of the shaft 30 with a hydrophilic coating agent or a hydrophobic coating agent. The coating agent may contain a drug or an additive.

Examples of the hydrophilic coating agent include hydrophilic coating agents made of hydrophilic polymers such as polyvinyl alcohol, polyethylene glycol, polyacrylamide, polyvinylpyrrolidone, and methyl vinyl ether-maleic anhydride copolymer, or any combination thereof.

Examples of the hydrophobic coating agent include polytetrafluoroethylene (PTFE), fluorinated ethylene propylene (FEP), perfluoroalkoxyalkane (PFA), silicone oil, hydrophobic urethane resin, carbon coating, diamond coating, diamond-like carbon (DLC) coating, ceramic coating, and substances having low surface free energy terminated with alkyl groups or perfluoroalkyl groups.

A drug may be retained on the outer surface of the straight tube portion 23 of the balloon 20. The drug is not particularly limited as long as it is a pharmacologically active substance, and examples of such drugs include pharmaceutically acceptable drugs, such as gene therapy agents, non-gene therapy agents, small molecules, and cells. In particular, when a catheter is used for the purpose of suppressing restenosis of a blood vessel after treatment in angioplasty, an anti-restenosis agent, such as an antiproliferative agent or an immunosuppressive agent, may be used as the drug. Examples of such drugs include paclitaxel, sirolimus (rapamycin), everolimus, and zotarolimus.

### 3. Tubular Member

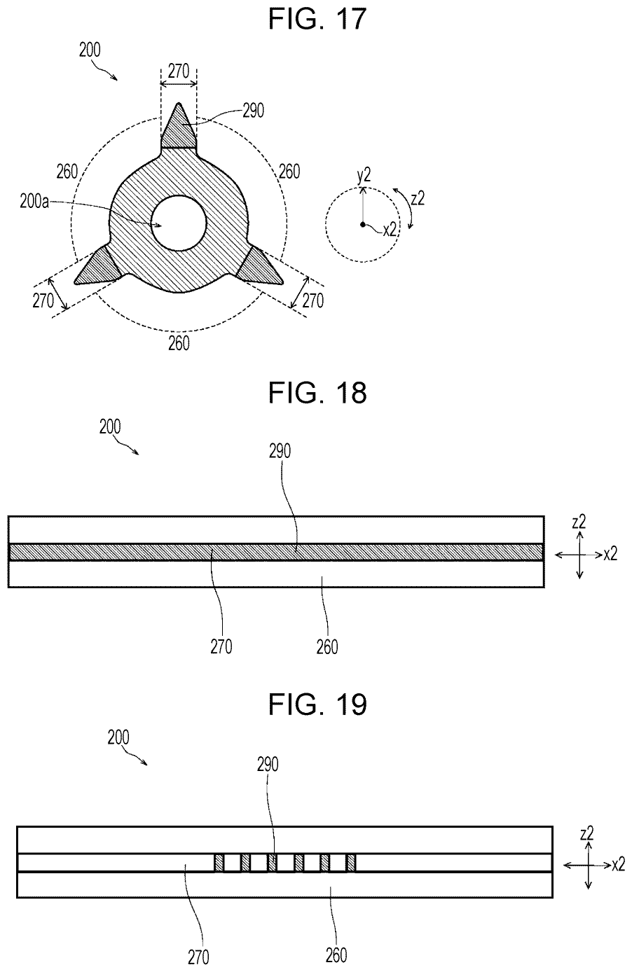

A tubular member according to one or more embodiments of the present invention will be described with reference to FIGS. 16 to 19. FIG. 16 is a perspective view of the tubular member according to one or more embodiments of the present invention. FIG. 17 is a cross-sectional view of the tubular member taken along line XVII-XVII of FIG. 16. FIG. 18 is a plan view of the tubular member illustrated in FIG. 17 as viewed from a protruding region side, and FIG. 19 is a plan view illustrating a modification of FIG. 18. Hatching in FIGS. 18 and 19 does not represent a cross section, but represents a colored portion. In the description of the tubular member, the same description as that of the balloon will be omitted.

As illustrated in FIG. 16, a tubular member 200 is made of a resin and has an inner lumen 200a. The tubular member 200 has a first end 201 and a second end 202, and extends in a longitudinal axis direction x2 from the first end 201 toward the second end 202. Similar to the balloon 20, the tubular member 200 has a radial direction y2 and a circumferential direction z2. For the resin forming the tubular member 200, reference can be made to the description of the resin forming the balloon 20.

The tubular member 200 has the inner lumen 200a extending in the longitudinal axis direction x2 and is used for manufacturing the balloon 20 for a balloon catheter by blow molding. To be specific, the balloon 20 can be manufactured by placing the tubular member 200 in a mold for manufacturing the balloon 20, introducing a fluid into the inner lumen 200a of the tubular member 200, and blow-molding the tubular member 200.

As illustrated in FIG. 17, the tubular member 200 has at least one protruding region 270 protruding outward in the radial direction y2 and extending in the longitudinal axis direction x2, and a non-protruding region 260 that is a region other than the protruding region 270. At least a part of the protruding region 270 is colored with a pigment or a dye. Since at least a part of the protruding region 270 is colored, the protruding portion 27 of the balloon 20 can be formed into a desired shape by cutting a colored portion 290 of the tubular member 200 before being molded into the balloon 20, or the colored portion 29 after the protruding region 270 of the tubular member 200 has been molded into the protruding portion 27 of the balloon 20 and the colored portion 290 of the protruding region 270 has been formed in the colored portion 29 of the protruding portion 27.

As illustrated in FIG. 17, a plurality of protruding regions 270 may be provided in the circumferential direction z2, or, although not illustrated, only one protruding region 270 may be provided in the circumferential direction z2. The number of the protruding regions 270 in the circumferential direction z2 may be 1 or more, 2 or more, 3 or more, 4 or more, or 6 or more, and may be 20 or less, 15 or less, or 10 or less. In the case where a plurality of protruding regions 270 are provided in the circumferential direction z2, the plurality of protruding regions 270 may be separated from each other in the circumferential direction z2, and may be arranged at substantially equal intervals in the circumferential direction z2. The distance between the protruding regions 270 separated from each other, that is, the length in the circumferential direction z2 of the non-protruding region 260, may be greater than the maximum length of the protruding regions 270 in the circumferential direction z2.

As illustrated in FIGS. 18 and 19, the protruding region 270 may be provided on the outer surface of the tubular member 200 so as to extend in a ridge-like manner in the longitudinal axis direction x2. This makes it easy to form the protruding portion 27 of the balloon 20.

As illustrated in FIG. 18, the protruding region 270 may be colored continuously in the longitudinal axis direction x2. This makes it easy to cut the tubular member 200 at any position in the longitudinal axis direction x2 by a laser.

Alternatively, as illustrated in FIG. 19, the protruding region 270 may be intermittently colored in the longitudinal axis direction x2. In this case, a portion desired to be cut in the protruding region 270 can be colored beforehand, so that positioning at the time of cutting is facilitated.

### 4. Method for Manufacturing Balloon Catheter

The method for manufacturing a balloon catheter according to one or more embodiments of the present invention includes a step of preparing the above-described tubular member 200, a step of cutting at least a part of the colored portion 290 of the tubular member 200 by a laser, and a step of manufacturing, by blow-molding the tubular member 200, the balloon 20 that includes the protruding portion 27 protruding outward in the radial direction y1 and extending in the longitudinal axis direction x1 and in which the protruding portion 27 has the at least one recess 28.

The laser to be used is not particularly limited, and examples thereof include a carbon dioxide laser, a fiber laser, an ultrashort pulse laser, and a YAG laser. In the method for manufacturing a balloon catheter according to one or more embodiments of the present invention, since the tubular member 200 that is molded into the balloon 20 has the colored portion 290, the colored portion 290 can be cut by absorbing even a low-output laser beam. Thus, efficient processing can be performed, and damage to an unintended portion can be prevented.

By using a laser to cut the protruding portion 27, it is possible to perform fine processing that is substantially the same as the focused beam diameter of the laser, and therefore, it is possible to form the recess 28 by cutting the top portion 27t of the protruding portion 27.

If the colored portion 290 is continuously arranged in the longitudinal axis direction x2 in the protruding region 270, any position in the longitudinal axis direction x2 can be cut by a laser. Alternatively, when the colored portion 290 is intermittently arranged in the longitudinal axis direction x2 in the protruding region 270, it is easy to perform cutting at a desired position by arranging the colored portion 290 beforehand at a position desired to be cut. Further, it is possible to prevent a portion that is not colored from being damaged by the laser.

Cutting using a laser may be performed on the colored portion 290 of the tubular member 200 before being molded into the balloon 20 or may be performed on the colored portion 29 after the tubular member 200 has been blow-molded and the colored portion 290 of the tubular member 200 has been molded into the colored portion 29 of the balloon 20.

This application claims the benefit of priority based on Japanese Patent Application No. 2023-89133, filed on May 30, 2023. The entire disclosure of Japanese Patent Application No. 2023-89133, filed on May 30, 2023, is incorporated herein by reference in its entirety.

### REFERENCE SIGNS LIST

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.
10 balloon catheter
20 balloon for balloon catheter
21 proximal-side sleeve portion
22 proximal-side tapered portion
23 straight tube portion
24 distal-side tapered portion
25 distal-side sleeve portion
26 balloon main body
26A non-protruding region of balloon
27 protruding portion
27A protruding region of balloon
27b base portion of protruding portion
27t top portion of protruding portion
27S protruding portion segment
28 recess
29 colored portion of balloon
30 shaft
31 inner shaft
31a guidewire port
32 outer shaft
32d distal-side outer shaft
32p proximal-side outer shaft
40 hub
50 fluid injection portion
60 tip member
70 radiopaque marker
200 tubular member
200a inner lumen
201 first end
202 second end
260 non-protruding region of tubular member
270 protruding region of tubular member
290 colored portion of tubular member

1. A balloon for a balloon catheter comprising:
a balloon main body having an outer surface and an inner surface; and
a protruding portion protruding radially outward from the outer surface of the balloon main body and extending in a longitudinal axis direction of the balloon main body,
wherein the balloon has a protruding region including the protruding portion in a circumferential direction of the balloon main body, and a non-protruding region not including the protruding portion in the circumferential direction of the balloon main body, and
wherein at least a part of the protruding region is colored with a pigment or a dye.

2. The balloon for the balloon catheter according to claim 1, wherein the non-protruding region is not colored.

3. The balloon for the balloon catheter according to claim 2, wherein, in a cross section perpendicular to the longitudinal axis direction, the balloon main body in the protruding region is not colored.

4. The balloon for the balloon catheter according to claim 2, wherein the protruding portion has at least one recess, and the protruding region is continuously colored in the longitudinal axis direction.

5. The balloon for the balloon catheter according to claim 2, wherein the protruding portion has at least one recess, and at least a part of the recess is not colored.

6. The balloon for the balloon catheter according to claim 2, wherein the protruding portion has at least one recess, and at least a part of the protruding region other than the recess is not colored.

7. A balloon catheter comprising the balloon according to claim 1.

8. A tubular member having a lumen extending in a longitudinal axis direction and configured to be used for manufacturing a balloon for a balloon catheter by blow molding,
wherein the tubular member has a protruding region protruding radially outward and extending in the longitudinal axis direction and a non-protruding region that is a region other than the protruding region, and
wherein at least a part of the protruding region is colored with a pigment or a dye.

9. The tubular member according to claim 8, wherein the protruding region is continuously colored in the longitudinal axis direction.

10. The tubular member according to claim 8, wherein the protruding region is intermittently colored in the longitudinal axis direction.

11. A method for manufacturing a balloon catheter, the method comprising:
preparing a tubular member that has a protruding region protruding radially outward and extending in a longitudinal axis direction and a non-protruding region that is a region other than the protruding region, the tubular member including a colored portion colored with a pigment or a dye in at least a part of the protruding region;
cutting at least a part of the colored portion by a laser; and
manufacturing, by blow-molding the tubular member, a balloon for the balloon catheter including a protruding portion protruding radially outward and extending in the longitudinal axis direction, the protruding portion having at least one recess.

12. The method according to claim 11, wherein the colored portion is continuously arranged in the longitudinal axis direction in the protruding region.

13. The method according to claim 11, wherein the colored portion is arranged intermittently in the longitudinal axis direction in the protruding region.
